(19) United States
(12) Patent Application Publication
(10) Pub. No.: US 2006/0217752 A1
(43) Pub. Date: Sep. 28, 2006

(54) Hair trimmer

(76) Inventor: Yong Hoon Cho, Fullerton, CA (US)

Correspondence Address: Maria Erlinda C. Sarno, Esq., P.O. Box 1023, Artesia, CA, 90702, US

(21) Appl. No.: 11/092,476

(22) Filed: Mar. 28, 2005

(51) Int. Cl. A61B 17/32

(52) U.S. Cl. 606/184

(57) ABSTRACT

A hair trimming device, comprising a punch handle having a jaw end and a lever end, the jaw end including a protruding punch element having a cutting circular peripheral rim; a die handle having a jaw end and a lever end, the jaw end including a die element comprising a cavity for receiving the punch element and storing cut unwanted hair strands prior to discard; a pin pivotally connecting the punch handle and the die handle; and, a spring plate between the punch handle and the die handle to allow the jaws to approach or move away from each other upon application of pressure on the lever ends of the handles. The outside surface of the jaw section of the trimmer comprising the jaw ends of the punch and die handle is uniformly smooth and is shaped to conform with the body cavity where the device will be used.

This invention relates to a manually operable trimmer for cutting unwanted or unsightly hair found in sensitive body cavities such as the ears and the nose.

### BACKGROUND

Present trimmers uses reciprocating blades. An example is a hollow cylindrical fixed blade with a plurality of teeth on top of the blade which is coupled with an internal blade that rotates inside the cylindrical blade. The hair is cut as it passes the cutting edges of the teeth of the cylindrical fixed blade. Often times, these reciprocating blades are connected to a fan or vacuum for removing hair strands as they are cut from the nostrils or ears.

Another nose trimming device has a curved blade strip that is generally U-shaped that fits within a nose or ear cavity. The curved blade strip has its two ends attached to a base portion. U.S. Pat. No. 5,983,499. The blade is usually protected by a blade guard which protects the skin within the cavity from being scratched while exposing portions of the cutting edges of the blade for trimming the nostril hair as the cutting edges are stroked linearly, downwardly or laterally across the skin in the same manner as hair is trimmed by a shaver.

These devices are generally complex in structure, thereby resulting in a more costly hair trimming device. Additionally, these are also more difficult to manufacture.

It is therefore an object of this invention to provide a hair triming device that is simple in design and safe for use.

It is also an object of this invention to provide a hair trimming device that is affordable to most users.

It is a further object of this invention to provide a hair trimming device that will catch the unwanted hair trimmed from the body cavities after they are cut.

### SUMMARY

This invention relates to a hair trimming device, comprising a punch handle having a jaw end and a lever end, the jaw end including a protruding punch element having a cutting circular peripheral rim; a die handle having a jaw end and a lever end, the jaw end including a die element comprising a cylindrical cavity for receiving the punch element and storing cut unwanted hair strands prior to discard; a pin pivotally connecting the punch handle and the die handle; and, a spring plate between the punch handle and the die handle to allow the jaws to approach or move away from each other upon application of pressure on the lever ends of the handles. The punch handle may be shorter than the die handle and is preferably but not necessarily ergonometrically designed, that is, the lever ends of the handles are contoured to accommodate a hand while grasping on the handles. The spring plate is made of a resilient material. One type of a spring plate comprises a flat planar section and a curved section, the flat planar section connecting to an inner surface of one handle, either the punch handle or the die handle, and the curved section extending from the flat planar section to an inner surface of another handle when the hair trmmer is ready to use and retracting towards the same inner surface of the handle where the planar section is connected when the hair trier is not in use. Another type of spring plate comprises a horizontal flat strip having two ends forming an arc between the punch handle and the die handle, one end connecting to an inner surface of one handle and the other end connecting to an inner surface of another handle. The cylindrical cavity of the die element has a depth deeper than the protruding height of the punch element thereby forming a space between the peripheral rim of the punch element and the bottom end of the die element when the punch element is fully engaged with the die element. The space catches and stores cut unwanted hair strands while the hair is being trimmed prior to discard. The cylindrical cavity of the die element can have either a flat bottom or a rounded bottom. The punchjaw and the die jaw comprise a flat top and a flat bottom surface, rounded side edges, two lateral outward surfaces, the lateral outward surfaces having a flat section on one end proximal to the pivot pin, the flat section gradually transitioning into a rounded surface on an end opposite the flat section. These rounded surfaces of the punch jaw and the die jaw form a semispherical tip when the hair trimmer is in a closed position. When the punch jaw protrudes from and the cavity of the die jaw is carved from respective flat inner surfaces of the jaws, it is recommended to round the edges of the respective flat inner surfaces to protect the skin of a user from being scratched. Further, the entire outward surface of both the punch jaw and the die jaw, herein also referred to as jaw section, should be uniformly smooth and shaped to conform with a shape of a body cavity on which the trimmer will be used.

Other embodiments of the present invention will become readily apparent to those skilled in the art from the following detailed description, wherein it shows and describes only certain embodiments of the invention by way of illustration. As will be realized, the invention is capable of other and different embodiments and its several details are capable of modification in various other respects, all without departing from the spirit and scope of the present invention. Accordingly, the drawings and detailed description are to be regarded as illustrative in nature and not as restrictive.

### BRIEF DESCRIPTION OF THE DRAWING

Aspects of the present invention are illustrated by way of example, and not by way of limitation, in the accompanying drawings, wherein:

FIG. 1 is a plan view of the hair trimmer with the jaws in an open position having a first design of a plate spring.

FIG. 1A is a plan view of the hair trimmer with the jaws in a closed position having a first design of a plate spring.

FIG. 2 shows the parts or components of the hair trimmer.

FIG. 3 is an exploded cross sectional view of the jaws of the hair trimmer having a die element with a flat bottom cylindrical cavity and showing the relationship of the die element to the punch element.

FIG. 3A is an exploded cross sectional view of the jaws of the hair trimmer having a die element with a rounded bottom cylindrical cavity and showing the relationship of the die element to the punch element.

FIG. 4 is a plan view of the hair trimmer with the jaws in an open position having a second design of a plate spring with the arc directed downwards.

FIG. 4A is a plan view of the hair trimmer with the jaws in an open position having a second design of a plate spring with the arc directed upwards.

FIG. 5 is a plan view of the hair trimmer with the jaws engaged and in a closed position having the first design of the plate spring.

### DETAILED DESCRIPTION OF THE INVENTION

The detailed description represented herein is not intended to represent the only way or the only embodiment in which the claimed invention may be practiced. The description herein is provided merely as an example or examples or illustrations of the claimed invention and should not be construed as the only way or as preferred or advantageous over other embodiments or means of practicing the invention. Any means of trimming unwanted hair from sensitive body cavities with the use of a punch element engaging with a die element is within the scope of this invention. The detailed description includes specific details to provide a thorough understanding of the claimed invention and it is apparent to those skilled in the art that the claimed invention may be practiced without these specific details.

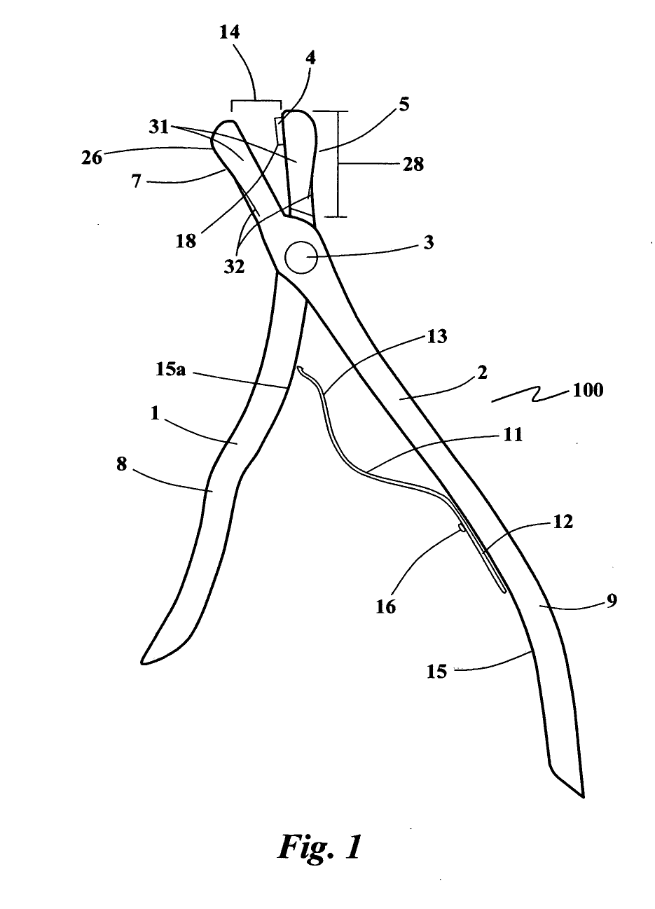

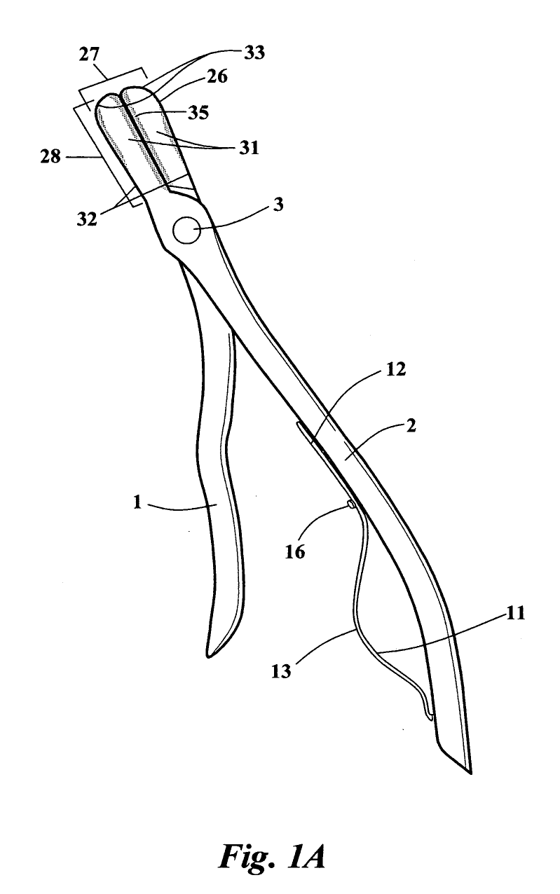

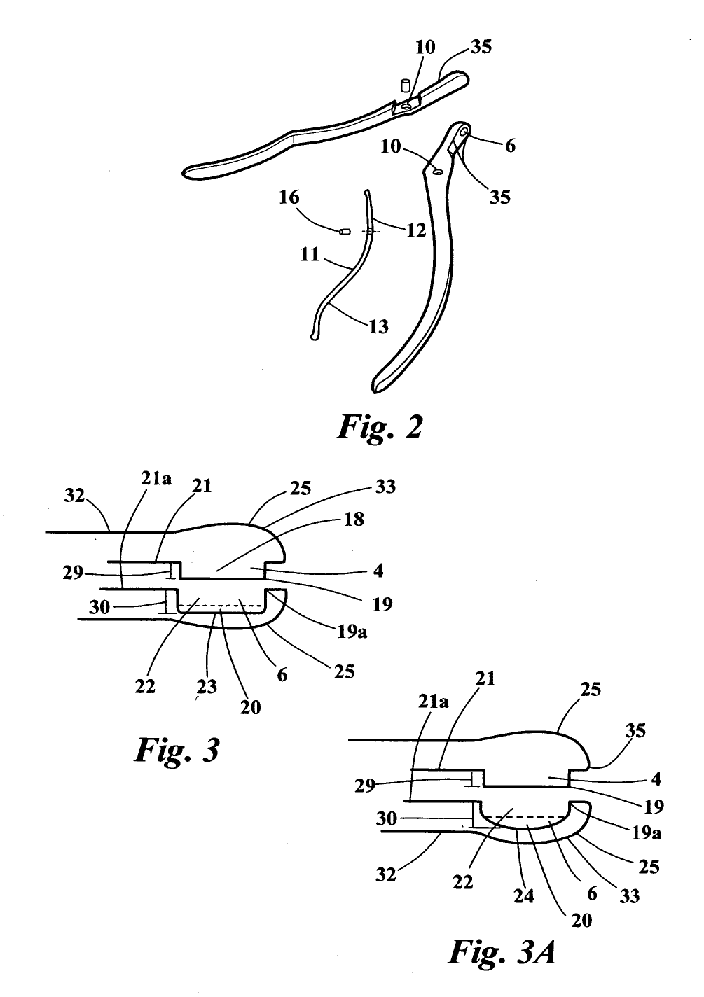

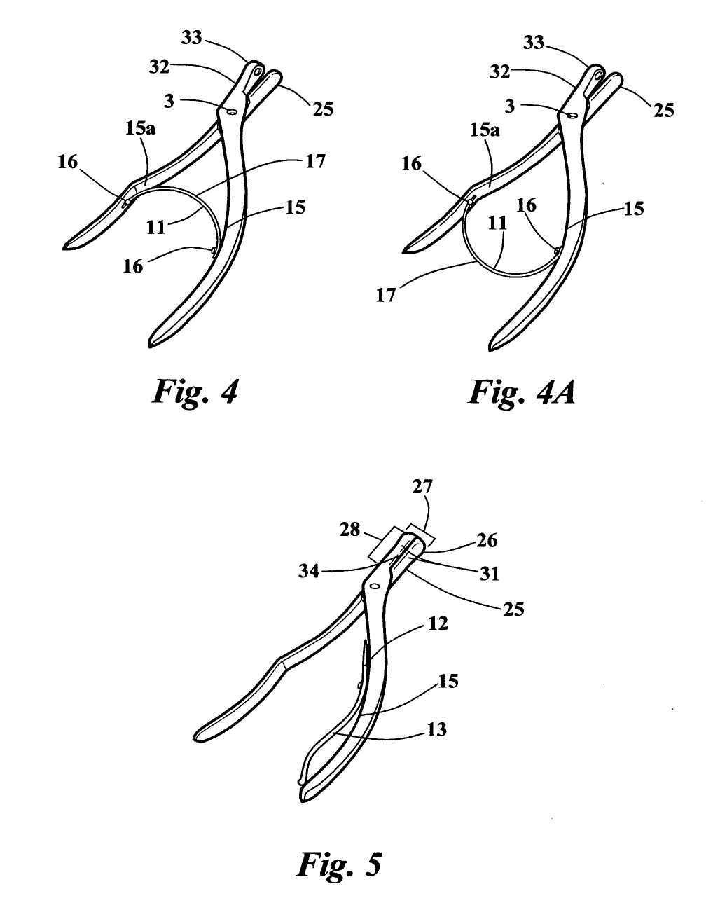

FIGS. 1 and 1A show a plan view of the claimed triming device 100. The hair trimmer comprises two handles 1 and 2 which are pivotally connected by a pivot pin or pivot fastener 3. There are different types of pivot pins or pivot fasteners and these are similar to those used for connecting the two handles of a scissor or a nail nipper. FIG. 2 shows the parts or components of the hair trimmer 100. One handle is identified herein as the punch handle 1 having a punch element 4 at one end of the handle, that end herein identified as the punch jaw 5. The other handle is identified herein as the die handle 2 having a die element 6 at one end of the handle, that end herein identified as the die jaw 7. The ends of the handles opposite the jaws function like a lever and is referred to herein as the lever end of the handle. The punch element 4 face directly opposite the die element 6 as shown in FIGS. 1 and 3. The lever end 8 of the punch handle 1 opposite the punch jaw 5 is preferably but not necessarily contoured to accommodate the four fingers and the lever end 9 of the die handle 2 opposite the die jaw 7 is arched outward to accommodate the inner surface of the thumb extending from beneath the thumb to the thumb as the hand grasps on the handles of the trimmer during usage. The described and shown shapes of the lever ends 8 and 9 opposite the jaws make pressing on the handles more comfortable as well as more effective in the manipulation of the trimmer. The punch handle 1 as shown in FIGS. 1, 4 and 4A, is preferably but not necessarily shorter than the die handle 2. The relationship of the lengths of the handles relative to each other lends itself to the position of the thumb in relation to the other four fingers as the hand grasps the hair trimmer while cutting the hair. The handles may be made of metal or hard plastic material. If made of metal, stainless steel is recommended to prevent the handles from rusting. The pivot pin 3 connects the handles by aligning the corresponding openings 10 located on both handles and inserting the pivot pin 3 through these openings. The openings 10 are located proximal to the jaw ends of the handles. Means for keeping the pivot pin in place while allowing the jaws to move forward or away from each other as the lever end of the handles are pressed or released are known.

To allow the jaws to approach or move away from each other only upon application of pressure on the lever ends of the handles, a plate spring 11 is connected between the two handles at a location below the pivot pin, proximal to the lever end of the handles as shown in FIGS. 1 and 4. There are usually two types of spring plate 11 used to accomplish the above said function. One design is a spring plate 11 having a flat planar section 12 and a curved section 13. As shown in FIGS. 1 and 1A, the flat planar section continuously extend to the curved section which curves out towards the other handle. The length of the curved section 13 depend upon the desired width 14 of the distance between the two opposing jaws when the hair trimmer is in the open position as shown in FIG. 1. The flat planar section 12 connects at the inner surface 15 of one handle by a screw or a pin 16. The screw or pin in this design serves as a pivot pin much like pivot pin 3. The screw or pin 16 allow the curved section 13 either to extend towards the inner surface 15a of the other handle or to retract towards the inner surface 15 of the same handle where the flat planar section 12 is connected by the screw or pin 16. The flat planar section may be connected to the inner surface of the punch handle or the die handle. In this design, the curved section 13 is free but touches on the inner surface 15a of the other handle when the trimmer is actuated and ready to use. When the trimmer is not in use or for storage, the curved section 13 is pivoted downwards towards the inner surface 15 of the same handle where the plate spring 11 is connected by the pin 16. The latter position will allow the two jaws to engage with each other and assume a closed position as shown in FIGS. 1A and 5. In the second design of the plate spring 11, the plate spring is a horizontal flat strip. One end of the strip is connected to the inner surface 15 of one handle while the other end is connected to the inner surface 15a of the other handle also by connectors like the screws and pins 16 thereby forming an arc 17. The arc may be directed upwards or downwards, respectively, as shown in FIGS. 4 and 4A. The plate spring 11 is made of a resilient material such as a thin strip of stainless steel metal to allow the two opposing jaws 5 and 7 to engage with each other, that is, the punch element is received into the die element when the lever ends of the handles 1 and 2 are pressed towards each other or to disengage when the pressure on the handles are released.

The punch element 4 on the punch handle 1 is a protruding metal cylinder 18 that is cut flat on the top to form a circular peripheral rim 19 that serves as a cutting edge. The punch element protrudes from a flat inner surface 21 of the punch jaw. The die element 6 on the die handle 2 is a corresponding cylindrical cavity carved out from a flat inner surface 21a of the die jaw to form a corresponding circular rim 19a that serves as the pair to the cutting edge on the circular peripheral rim 19 of the punch element. The cylindrical cavity 22 of the die element 6 perfectly aligns with the punch element 4 to enable the cavity 22 to receive or engage with the punch element 4 as the peripheral rim 19 of the punch element cuts the hair after contact with the peripheral rim 19a of the die element. The cylindrical cavity 22 may have a flat bottom 23 as shown in FIG. 3 or a rounded bottom 24 as shown in FIG. 3A. The depth and shape of the cavity 22 provides an important feature of the invention. The depth 30 of the cavity 22 is deeper than the protruding height 29 of the punch element to form a space 20 between the peripheral rim 19 of the punch element 4 and the bottom 23 and 24 of the cavity 22 when the punch jaw and the die jaw are fully engaged. The broken lines in FIGS. 3 and 3A show the location of the tip or peripheral rim of the punch element when fully inserted into the die element. Space 20 is underneath the broken lines. The space 20 of the cavity 22 catches the cut unwanted hair strands while the hair is being trimmed and stores these strands prior to discard. This prevents cut hair strands from scattering around its surroundings which is not only unsightly but requires the additional step of sweeping the surroundings such as the floor. Herein, the cut hair strands caught within the space 20 of the cavity 22 can be discarded into a trash receptacle for example, after the trimming of the hair.

Another important feature of the trimming device is the contour of the lateral outward surface 25 of the jaw ends 5 and 7. As shown in FIGS. 1, 1A, 4, 4A, and 5, the top 31 and bottom surfaces (not shown) of the punch jaw and the die jaw are flat. The flat surfaces of the jaws gradually transition into smooth rounded edges on the sides 26 which are edges common with the lateral outward surfaces 25 of the jaws. The lateral outward surface has a flat section 32 proximal to the screw or pin 3 which gradually transitions into a rounded surface 33 as shown in FIGS. 1A, 4 and 4A. When the punch jaw and the die jaw are fully engaged, that is, the hair trimmer 100 is in a closed position, the rounded surfaces of the two jaws assume a semispherical tip 27. The semispherical tip 27 may be fully rounded like a dome as shown in FIG. 5. It may also have a rounded break at the middle like the small letter ‘m’ which occurs when the edges 35 of the flat inner surfaces 21 and 21a which form the junction 34 between the die jaw and the punch jaw are also rounded to form a trimmer with a jaw section 28 shown in FIG. 1A. Additionally, it is recommended to have the entire outward surfaces of the punch jaw and the die jaw, collectively herein referred to as jaw section 28 of the trimmer 100 to be uniformly very smooth especially at the junction 34 between the die jaw and the punch jaw to avoid scratching the skin when the hair trimmer is introduced into the body cavity such as the nose, ear and the like. The shape of the jaw section 28 of the hair trimmer 100 may vary but this should conform or be compatible with the shape of the body cavity where the hair trier will be used. Likewise, the shape of the punch element and its corresponding die element, while illustrated herein as cylindrical, may have different geometric shapes.

The hair trimmer 100 is generally used by grasping and slightly pressing on the handles of the trimmer and introducing the device into the body cavity in a semi open position. The width of the opening 14 between the die and the punch element is at the discretion of the user. The hair or hairs to be cut are placed between the punch element 4 and the die element 6. Cutting is done by fully pressing on the lever ends of handles 1 and 2 which consequently cuts the hair as the peripheral rim 19 of the punch element engages with peripheral rim 19a of the die element. The cut hair strands fall into the space 20 of the cavity 22 until they are discarded.

While the embodiments of the present invention have been described, it should be understood that various changes, adaptations, and modifications may be made therein without departing from the spirit of the invention and the scope of the claims.

I claim:

1. A hair trimming device, comprising:
a punch handle having a jaw end and a lever end, the jaw end including a protruding punch element having a cutting circular peripheral rim;
a die handle having a jaw end and a lever end, the jaw end including a die element comprising a cylindrical cavity for receiving the punch element and storing cut unwanted hair strands prior to discard;
a pin pivotally connecting the punch handle and the die handle; and,
a spring plate between the punch handle and the die handle to allow the jaws to approach or move away from each other upon application of pressure on the lever ends of the handles.

2. The device of claim 1 wherein the punch handle is shorter than the die handle.

3. The device of claim 1 wherein the lever ends of the handles are contoured to accommodate a hand while grasping on the handles.

4. The device of claim 1 wherein the spring plate is made of a resilient material.

5. The device of claim 1 wherein the spring plate comprises a flat planar section and a curved section, the flat planar section connecting to an inner surface of one handle, either the punch handle or the die handle, the curved section extending from the flat planar section to an inner surface of another handle when the hair trimmer is ready to use and retracting towards the same inner surface of the handle where the planar section is connected when the hair trimmer is not in use.

6. The device of claim 1 wherein the spring plate is a horizontal flat strip having two ends forming an arc between the punch handle and the die handle, one end connecting to an inner surface of one handle and the other end connecting to an inner surface of another handle.

7. The device of claim 1 wherein the cylindrical cavity of the die element has a depth deeper than a protruding height of the punch element thereby forming a space between the peripheral rim of the punch element and a bottom end of the cavity of the die element when the punch element is fully engaged with the die element.

8. The device of claim 7 wherein the space catches and stores cut unwanted hair strands while the hair is being trimmed prior to discard.

9. The device of claim 1 wherein the cylindrical cavity of the die element has a flat bottom or a rounded bottom.

10. The device of claim 1 wherein the punch jaw and the die jaw comprise a flat top and a flat bottom surface, rounded side edges, two lateral outward surfaces, the lateral outward surfaces having a flat section on one end proximal to the pivot pin, the flat section gradually transitioning into a rounded surface on an end opposite the flat section.

11. The device of claim 10 wherein the rounded surfaces of the punch jaw and the die jaw form a semispherical tip when the hair trimmer is in a closed position.

12. The device of claim 1 wherein the punch element protrudes from a flat inner surface of the punch jaw.

13. The device of claim 12 wherein the flat inner surface has rounded edges.

14. The device of claim 1 wherein the cavity of the die element is carved out from a flat inner surface of the die jaw.

15. The device of claim 14 wherein the flat inner surface has rounded edges.

16. The device of claim 1 wherein an entire outward surface of both the punch jaw and the die jaw are uniformly smooth to avoid scratching the skin of a user.

17. The device of claim 1 wherein a jaw section including the jaw ends of the punch jaw and the die jaw of the hair trimmer is shaped to conform with a shape of a body cavity on which the device will be used.

18. A hair trimming device, comprising:
a punch handle having a jaw end and a lever end, the jaw end including a protruding punch element having a cutting circular peripheral rim;
a die handle having a jaw end and a lever end, the jaw end including a die element comprising a cylindrical cavity for receiving the punch element, the cylindrical cavity having a depth deeper than a height of the protruding punch element resulting in a space between the peripheral rim of the punch element and a bottom end of the die element when the punch element is fully engaged with the die element, the space storing cut unwanted hair strands prior to discard;
a pin pivotally connecting the punch handle and the die handle;
a spring plate between the punch handle and the die handle to allow the jaws to approach or move away from each other upon application of pressure on the lever ends of the handles; and,
a jaw section including the jaw ends of the punchjaw and the die jaw having a uniformly smooth rounded outward surface conforming to a shape of a body cavity.
